(19) United States
(12) Patent Application Publication
(10) Pub. No.: US 2021/0287810 A1
(43) Pub. Date: Sep. 16, 2021

(54) MEDICAL SYSTEM FOR PROVIDING A TREATMENT RECOMMENDATION

(71) Applicant: Drägerwerk AG & Co. KGaA, Lübeck (DE)

(72) Inventors: Frank FRANZ, Lübeck (DE); Stefan SCHLICHTING, Lübeck (DE); Simon GISCH, Lübeck (DE); Hinrich ALTHOFF, Lübeck (DE)

(21) Appl. No.: 17/196,096

(22) Filed: Mar. 9, 2021

(30) Foreign Application Priority Data
Mar. 10, 2020 (DE) 102020001563.8

(51) Int. Cl. G16H 50/70; G16H 10/60; G16H 50/20; G16H 40/67; G16H 40/40; G06F 16/11

(52) U.S. Cl. CPC G16H 50/70; G16H 10/60; G06F 16/113; G16H 40/67; G16H 40/40; G16H 50/20

(57) ABSTRACT

A medical system (100), for providing a treatment recommendation (105), includes an archiving module (110) providing a structured patient archive (130) with a respective patient data set (132) for each of several patients. The patient data set indicates a course of treatment (520) and a treatment result (530) of the corresponding patient. At least one digital patient profile (134), indicating features of the treatment course (542, 542′, 542″) and treatment result (544), is assigned to the patient data set. A processing module receives current patient data (142) recorded by an external medical device (140) and determines a current patient data set (124) with a corresponding current digital patient profile (125) and compares the current digital patient profile with the stored digital patient profiles of the archiving module and determines the treatment recommendation based on this comparison and outputs a treatment signal (128) that indicates the determined treatment recommendation.

### CROSS REFERENCE TO RELATED APPLICATIONS

This application claims the benefit of priority under 35 U.S.C. § 119 of German Application 10 2020 001 563.8, filed Mar. 10, 2020, the entire contents of which are incorporated herein by reference.

### TECHNICAL FIELD

The present invention pertains to a medical system for providing a treatment recommendation. The present invention further pertains to a process for providing a treatment recommendation.

### TECHNICAL BACKGROUND

It is known that an actual course of treatment is compared with a predefined expected course of treatment during a medical treatment. A change in the current treatment of the patient can be triggered on the basis of such a comparison in an automated manner or on the basis of medical expertise. The predefined expected course of treatment may be predefined, for example, by a central guideline, for example, by a professional society.

Such a guideline can typically be followed rarely without any deviation, because each patient has a body that is to be taken into consideration individually and has a correspondingly individual course of treatment. It is known against this background that an automated treatment recommendation is issued based on such a guideline.

Thus, US 2019/0057774 A1 describes a device trained by means of machine learning, which issues a treatment recommendation for a patient to be currently treated on the basis of a guideline published by medical specialists and thereby assists the attending physician in the treatment of a patient.

### SUMMARY

An object of the present invention is to provide an improved treatment recommendation, and in particular an especially reliable treatment recommendation, for the treatment of a patient.

A medical system for providing a treatment recommendation is provided according to the present invention for accomplishing this object. The medical system comprises an archiving module and a processing module.

The archiving module is configured to provide a structured patient archive, in which a respective patient data set is stored for each patient of a plurality of patients, wherein the respective patient data set indicates a course of treatment and a treatment result of the corresponding patient. At least one digital patient profile, which indicates features of the course of treatment and of the treatment result, is assigned to the patient data set of a respective patient.

The processing module has a medical device interface and an archiving module interface, wherein the processing module is configured to receive current patient data recorded by an external medical device via the medical device interface and to determine from this a current patient data set with a corresponding current digital patient profile. Furthermore, the processing module is configured to compare the current digital patient profile with the plurality of stored digital patient profiles by a communication with the archiving module via the archiving module interface and to determine the treatment recommendation based on this comparison, taking into consideration the treatment results indicated by the stored digital patient profiles. Finally, the processing module is further configured to output a treatment signal, wherein said treatment signal indicates the determined treatment recommendation.

It was found within the framework of the present invention that by analyzing earlier courses of treatment and corresponding earlier treatment results, information can be systematically collected on courses of treatment, which are comparable to a current treatment of a patient. Such comparable courses of treatment may be helpful when making a decision about the currently best treatment strategy. Features of the course of treatment and of the treatment result are combined to this end into a digital patient profile in order to make it thereby possible automatically to detect comparable courses of treatment of corresponding patients.

The medical system according to the present invention makes it advantageously possible to compare a current treatment situation of a patient with a plurality of earlier treatment situations, which are similar according to the patient profile. A current treatment recommendation can be inferred now from the course of treatment analyzed for an earlier treatment situation and from the treatment result that occurred in that case. In particular, possible errors, which have led to a disadvantageous treatment result in a similar treatment situation, can be avoided thereby during the current treatment.

According to the present invention, the digital patient profiles have only individual features of the course of treatment documented within the framework of the respective patient data set and of the corresponding treatment result. Data can advantageously be compared with one another more rapidly by the comparison of the patient profiles than when complete patient data sets are compared with one another. Furthermore, the use of a patient profile makes it advantageously possible to reduce the large number of features within a typical patient data set to features that are especially relevant for the characterization of the treatment. The data structure of the digital patient profiles and preferably the type of the detected features within the digital patient profiles are predefined in order to guarantee comparability of the digital patient profiles.

The treatment recommendation indicates according to the present invention a recommendation for the future treatment of the current patient, for example, within the framework of a setting recommendation for a medical device connected or to be connected to the patient. The issuing of a treatment recommendation ensures that a real recipient, for example, medical staff, will check the recommendation before it is executed by a device. Risks due to technical errors, as they could occur, for example, during a fully automated treatment, can be avoided thereby.

The structured patient archive comprises an assignment between a course of treatment of an individual, concrete patient and the corresponding treatment result. In the sense of such an assignment, the patient archive is structured. Provisions can now be made according to the present invention within the framework of the patient archive not to make possible an unambiguous assignment to personal data of a concrete patient. As a result, the medical system according to the present invention can advantageously be adapted to regional data protection regulations.

The archiving module and the processing module may be arranged within a common device. In particular, the archiving module and the processing module may be configured by means of a common processor, wherein the processing steps are divided at least at the software level into processing steps of the archiving module and into processing steps of the processing module. The archiving module preferably comprises a memory, in which the structured patient archive is provided. However, the archiving module may also be configured according to the present invention such that it accesses an external memory with the structured patient archive. In particular, the detection of the structured patient archives and/or of patient data for the structured patient archive may also take place outside the medical system according to the present invention.

The archiving module and the processing module may be arranged within a common housing. As an alternative, the archiving module and the processing module may be configured and arranged as modules located at a distance from one another. The connection between the archiving module and the processing module may be configured as a cable-based or wireless connection.

The medical device interface and the archiving module interface may be configured each for a wireless or cable-based connection. The precise configuration of such an interface for prior-art cables, e.g., Ethernet cable, or prior-art wireless connections, e.g., WLAN, Bluetooth, BLE or ZigBee, is known and will not therefore be described in detail below.

Preferred embodiments of the medical system according to the present invention will be described below.

In a preferred embodiment, the determination of the treatment recommendation comprises an analysis of the stored patient data sets in which the digital patient profile has a sufficient agreement with the current digital patient profile. It is advantageously ensured in this embodiment that the features of the course of treatment of an earlier patient, which are detected within the digital patient profile, have a sufficient agreement with the course of treatment of the patient currently being treated. A sufficient agreement is, for example, a similarity in a predefined minimum number of features or a similarity for a predefined selection of concrete selection features, for which an agreement is necessary. A similarity of features is, for example, a deviation of a measured value for a feature between the earlier patient and the current patient below a predefined threshold value. Only the patient data sets that have, on the whole or at least for concretely predefined selection features, a high level of similarity to the current patient data set are advantageously analyzed in this embodiment. It is made possible hereby that only comparable treatment situations are used for a comparison within the processing module.

In an especially preferred embodiment, the analysis of the stored patient data sets in which the digital patient profile has a sufficient agreement with the current digital patient profile comprises an analysis of a relationship between the respective course of treatment indicated by the corresponding patient data set and the respective treatment result indicated by the corresponding patient data set. It is advantageously ensured in this embodiment that when determining the treatment recommendation, the processing module according to the present invention detects the patient data sets that show a sufficient agreement with the current digital patient profile and have led to an unfavorable treatment result as a treatment that is not to be repeated and/or it detects patient data sets that show a sufficient agreement with the current digital patient profile and have led to a favorable treatment result as a recommendable treatment. An unfavorable treatment result may be, for example, death, an exacerbation of the health status and/or a lack of onset of improvement of the health status. A favorable treatment result may be, for example, recovery, improvement of the health status and/or a lack of onset of an exacerbation of the health status. A specific detection of favorable treatment courses and/or of unfavorable treatment courses are thus made advantageously possible in this embodiment. An especially reliable treatment recommendation can be determined hereby.

In another embodiment, each stored patient data set comprises at least one operating state of an electromedical device assigned to the corresponding patient. The stored treatment course advantageously also comprises in this embodiment the at least one operating state of the assigned electromedical device. The treatment recommendation preferably likewise comprises here a desired operating state of the assigned electromedical device. Thus, the medical staff treating the patient can find in the issued treatment recommendation concrete information as to actions to be taken, on the basis of which the staff can make decisions using their medical expertise. In a variant of this embodiment, the respective patient data set comprises a plurality of operating states of the assigned electromedical device. In an alternative or additional variant of the above embodiment, the respective patient data set comprises at least one operating state of a plurality of electromedical devices assigned to the corresponding patient. In an alternative embodiment, each stored patient data set comprises exclusively physiological measured values, which were recorded during the treatment of the corresponding patient.

In another advantageous embodiment, respective time periods of a treatment step are assigned to the course of treatment within a respective stored patient data set. To determine a treatment recommendation, an analysis of the assigned time periods is preferably carried out to determine a treatment recommendation. The provision of taking respective time periods of a treatment step into consideration makes it advantageously possible to assign the current treatment situation of the patient currently being treated to a concrete time within the time period of a respective patient data set. The current treatment situation can thus be assigned to different phases of a course of treatment of a patient treated earlier. A course of treatment of an earlier treatment can therefore also be compared in this embodiment with the current treatment situation when a start situation of the earlier treatment was entirely different from the currently present start situation. In particular, the provision of assigned time periods makes it possible to obtain information on a preferred duration of treatment or on a plurality of durations of treatment, especially for different treatments, within the defined treatment recommendation. Finally, the provision of respective time periods of a treatment step within the patient data set makes possible a specific analysis of the treatment steps within a respective stored patient data set that occurred chronologically after the current treatment situation.

In another advantageous embodiment, the treatment recommendation comprises a treatment warning. A warning can be issued in this embodiment by the processing module according to the present invention against a concrete future treatment, especially against a continuation of a current treatment. Thus, a treatment recommendation is detectably issued in a variant of this embodiment precisely when a current treatment, for example, a current setting of an electromedical device connected to the patient, led to an unfavorable treatment result within the framework of an earlier course of treatment or within the framework of a number of earlier courses of treatment for the current treatment situation. The medical system according to the present invention can thus advantageously be used as a warning system.

In an especially preferred embodiment, the treatment recommendation comprises an operating state of an electromedical device assigned to the corresponding patient, which operating state is to be used, a relative change in the operating state of an electromedical device assigned to the corresponding patient, an electromedical device to be assigned to the corresponding patient, a duration of a treatment to be carried out and/or a frequency of a treatment to be carried out. The treatment recommendation according to this embodiment advantageously gives a concrete recommendation for the attending medical staff. This makes it possible for the medical staff to make a conscious decision on whether a treatment proposed according to the treatment recommendation is desired or not desired. It is ensured hereby that there is a conscious decision concerning the desired treatment. Furthermore, it is advantageously ensured that at least one treatment scenario is proposed to the attending medical staff, as a result of which an inadvertent false assessment of the current situation of the patient, especially due to important treatment-related information being overlooked, can be avoided. For example, a proposed treatment scenario, of which the medical staff has not yet thought at all, would support a reassessment of the current situation of the patient by the medical staff

In an especially preferred embodiment, the archiving module is connected to an external patient management system such that patient data sets of treated patients are continuously stored in the structured patient archive. It is ensured in this embodiment that new patient data are continuously stored in the patient archive. As a result, patient data are continuously adapted to new treatment methods and to the treatment by new medical devices. Only patient data of patients of one hospital or of a region of hospitals are stored continuously within the structured patient archive. As a result, the stored patient data sets with the respective course of treatment are especially representative of the average patients of a concrete hospital or of a concrete region, which is advantageous especially in view of regional disease symptoms. Finally, the use of a local patient archive, for example, of a patient archive for a concrete hospital, is advantageous due to the fact that the stored courses of treatment depict the treatment with the medical devices that are indeed available in the hospital in which the treatment is currently being carried out.

In another embodiment, the medical system comprises, furthermore, an input module, which is configured to receive user information via an input interface, wherein the user information comprises patient data to be stored in the current patient data set, a desired treatment result and/or a patient group to be assigned to the current patient data set. User information according to this embodiment makes possible an especially rapid filtering of relevant patient profiles within the archiving module for the comparison according to the present invention within the processing module. Thus, a large number of stored patient data sets can be reduced to a smaller subset of relevant patient data sets that are meaningful for a comparison of digital patient profile and current digital patient profile. Patient data that are to be stored are, for example, data detected at the time of admission of a patient, such as age, body weight and/or height. A patient group to be assigned typically comprises patients whom experience has shown to respond similarly to a treatment, for example, patients within a predefined age range, patients with a common clinical picture, e.g., diabetes, patients of a common risk group, e.g., smokers, and/or patients with characteristic physical features. The input module is connected to the archiving module and/or to the processing module. Such a connection is a cable-based or wireless connection, especially a connection via an Ethernet cable or via a WLAN connection. The input module preferably comprises a network card in order to communicate with the processing module and/or with the archiving module via a network of the medical facility, especially of a hospital.

In another embodiment, the medical system has, furthermore, a testing module, which is configured to receive the treatment signal and to compare the indicated treatment recommendation with device data of at least one electromedical device assigned to the corresponding patient and to determine a checked treatment recommendation on the basis of this comparison, wherein the testing module is further configured to output a checked treatment signal, wherein the checked treatment signal indicates the checked treatment recommendation. It can be ensured in this embodiment that the electromedical device used makes possible a treatment corresponding to the treatment recommendation. Furthermore, a comparison can be carried out between device data within the treatment recommendation and the device data of the electromedical device used, so that, for example, a recommendation for a treatment to be carried out by an electromedical device different from the electromedical device currently being used is avoided. Furthermore, it can be ensured by a testing module in a variant of this embodiment that the treatment recommendation does not violate a guideline set by a medical specialist for the electromedical device being used. The testing module is connected to the processing module. Such a connection is a cable-based connection or has a wireless configuration, being configured especially as a connection via an Ethernet cable or via a WLAN connection. The testing module preferably comprises a network card in order to communicate with the processing module via a network of the medical facility, especially of a hospital. The testing module is preferably configured to communicate, furthermore, with an output screen for outputting the checked treatment recommendation, especially via a network of the medical facility.

In another, especially preferred embodiment, the medical system has, furthermore, an output module, which is configured to output the treatment signal and/or the checked treatment signal to the at least one electromedical device assigned to the corresponding patient. As a result, a treatment of the patient can be carried out in an automated manner corresponding to the treatment recommendation and/or to the checked treatment recommendation. The assigned electromedical device especially outputs the treatment recommendation in a recognizable manner to the medical staff, so that a regulation or control of the electromedical device is carried out ultimately by the medical staff corresponding to the treatment recommendation. In an alternative or additional embodiment, the output module outputs the treatment recommendation and/or the checked treatment recommendation via a user interface, especially via a display. As a result, the treatment recommendation is recognizable for the attending medical staff. In an exemplary variant, the user interface is a display, especially a display of a portable device, e.g., of a Smartphone. The medical system according to the present invention is preferably configured to communicate with a nearest mobile output module, especially with a mobile output module registered within the medical system, via a wireless connection, e.g., a WLAN, Bluetooth, BLE or ZigBee connection. As a result, the attending person can receive the checked treatment signal by means of a mobile output module or output device carried along, e.g., by means of a Smartphone, tablet or Notebook, and detect the corresponding treatment recommendation and/or the checked treatment recommendation.

The treatment recommendation may be outputted by an optical display, especially by a pictographic representation of a treatment to be carried out, e.g., of an electromedical device to be used. Furthermore, the treatment recommendation may comprise an operating state of an electromedical device connected to the patient being treated, which operating state is to be used, or a change in the operating state of this electromedical device or of a plurality of electromedical devices, which change in the operating state is to be used.

According to another aspect of the present invention, a process for providing a treatment recommendation is proposed for accomplishing the above-mentioned object. The process according to the present invention has the following steps:
provision of a structured patient archive, in which a respective patient data set is stored for each patient of a plurality of patients, wherein the respective patient data set indicates a course of treatment and a treatment result of the corresponding patient, and wherein at least one digital patient profile, which indicates features of the course of treatment and of the treatment result, is assigned to the patient data set of a respective patient;
reception of current patient data recorded by a medical device;
determination of a current patient data set with a corresponding current digital patient profile from the recorded current patient data;
comparison of the current digital patient profile with the plurality of stored digital patient profiles; and
determination of the treatment recommendation based on the comparison, taking into consideration the treatment results indicated by the stored digital patient profiles, and outputting of a corresponding treatment signal, wherein the treatment signal indicates the determined treatment recommendation.

The process according to the present invention has the same advantages as the medical system according to the present invention. In particular, the process according to the present invention advantageously makes possible an analysis of past courses of treatment in a similar treatment situation of a patient in order for a current treatment to lead to a favorable treatment result with as high a certainty as possible.

The process steps may be carried out by a common device, for example, via a common processor. The process steps may also be carried out, as an alternative, partially on different devices, preferably on different devices connected to one another.

In an especially preferred embodiment, the process according to the present invention has, furthermore, the following steps:
reception of the treatment signal and comparison of the indicated treatment recommendation with device data of at least one electromedical device assigned to the corresponding patient; and
determination of a checked treatment recommendation based on the preceding comparison and outputting of a corresponding, checked treatment signal, wherein the checked treatment signal indicates the checked treatment recommendation.

The present invention shall now be explained in more detail on the basis of advantageous embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a medical system according to the present invention;

FIG. 2 is a schematic view of a second exemplary embodiment of the medical system according to the present invention;

FIG. 3 is a schematic view of a third exemplary embodiment of the medical system according to the present invention;

FIG. 4 is a schematic view of a fourth exemplary embodiment of the medical system according to the present invention;

FIG. 5 is a schematic view of a structured patient archive for the medical system according to an exemplary embodiment according to the present invention;

FIG. 6 is a schematic view of a comparison of a current digital patient profile with a plurality of stored digital patient profiles for the medical system according to an exemplary embodiment according to the present invention; and

FIG. 7 is a flow chart of an exemplary embodiment of a process according to another aspect of the present invention.

### DESCRIPTION OF PREFERRED EMBODIMENTS

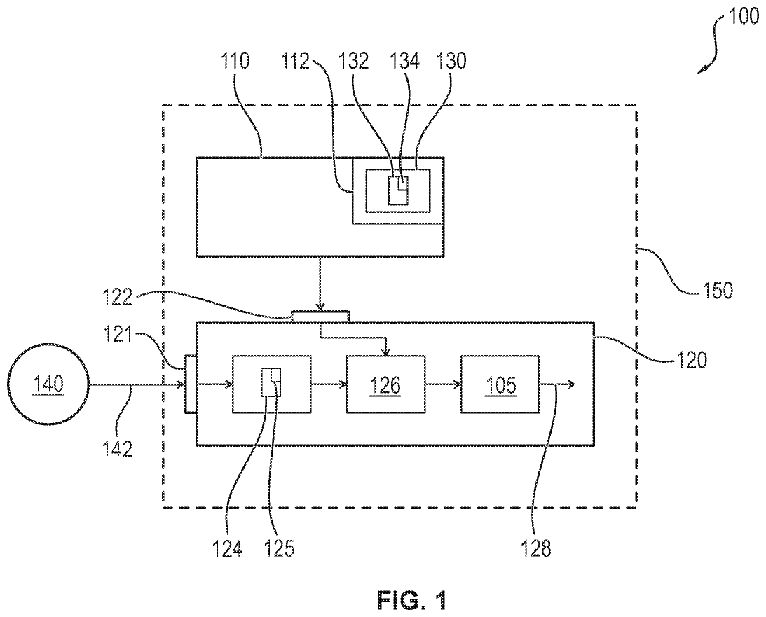

Referring to the drawings, FIG. 1 shows a schematic view of a first exemplary embodiment of a medical system 100 according to the present invention.

The medical system 100 is configured to provide a treatment recommendation 105 and has an archiving module 110 and a processing module 120.

The archiving module 110 is configured to provide a structured patient archive 130, in which a respective patient data set 132 is stored for each patient of a plurality of patients. The plurality of patients is preferably an already treated plurality of patients, for whom a corresponding treatment result and a complete course of treatment are already available. The respective patient data set 132 indicates here the course of treatment and the treatment result of the corresponding patient. The data within the patient data set 132 are preferably represented in a concise form in order to make possible the lowest possible storage effort possible. At least one digital patient profile 134, which indicates features of the course of the treatment and of the treatment result, is assigned according to the present invention to the patient data set 132 of a respective patient. The features are preferably especially essentially features of the course of the treatment and of the treatment result, which characterize the course of the treatment and the treatment result in a highly concise form. The patient profile 134 may be stored together with the patient data set 132 within a memory 112. In particular, the patient data set 132 and the patient profile 134 may be stored within a common file. In the example shown, the archiving module 110 comprises the memory 112. The data in the memory 112 are regularly complemented by new patient data sets in the exemplary embodiment shown. The fact that new patient data sets 132 are regularly taken into account makes it possible to take into consideration new treatment methods and new medical devices within the structured patient archive 130.

The processing module 120 has at least one medical device interface 121 and an archiving module interface 122. The medical device interface 121 makes possible a communication with at least one external medical device 140. This communication may take place in a cable-based or wireless manner. In particular, the communication may take place via an Ethernet cable, a WLAN connection, a Bluetooth connection and/or a BLE connection. The archiving module interface 122 makes possible a communication with the archiving module 110. This communication may take place in a cable-based or wireless manner. In particular, the communication may take place via a WLAN connection, a Bluetooth connection and/or via a BLE connection. The processing module 120 is configured to receive the current patient data 142 recorded by the at least one external medical device 140 via the medical device interface 121 and to determine therefrom a current patient data set 124 with a corresponding current digital patient profile 125. Furthermore, the processing module 120 is configured to compare the current digital patient profile 125 with the plurality of stored digital patient profiles 134 by a communication with the archiving module 110 via the archiving module interface 122 and to determine a treatment recommendation 105 on the basis of this comparison 126. The treatment recommendation 105 may indicate here a treatment to be used and/or the necessity to change a current treatment of the patient. In particular, the treatment recommendation 105 may indicate an operating state to be used and/or a relative change in the operating state of the electromedical device 140 connected to the patient. Finally, the treatment recommendation 105 may comprise, in addition or as an alternative, an electromedical device to be newly assigned to the corresponding patient, a duration of the treatment to be carried out and/or a frequency of a treatment to be carried out.

Furthermore, the processing module 120 is configured to output a treatment signal 128, wherein the treatment signal 128 indicates the treatment recommendation 105 determined.

The treatment signal 128 may be analyzed, especially analyzed internally by the medical system 100 according to the present invention or be outputted directly to the electromedical device 140 for the further processing or be outputted directly to a display device that is available to the user of the medical system 100. The outputting of the treatment signal 128 is carried out in a wireless or cable-based manner. In particular, the corresponding communication may take place via an Ethernet cable, a WLAN connection, a Bluetooth connection and/or a BLE connection.

The treatment recommendation 105 may be outputted according to the present invention as an output to a user of the medical system 100 for information in order to advantageously affect thereby a treatment of the patient by the user, which will be carried out in the future. As an alternative or in addition, the treatment recommendation 105 may trigger a control of the electromedical device 140 directly and thereby act directly on the treatment of the patient. An approval signal, especially a digital approval signal, especially an approval signal signed by medical staff, is preferably waited for before a direct action on the treatment of the patient, before an automated treatment is continued. An incorrect treatment based on technical problems can be avoided hereby. An input device for receiving the approval signal may be provided at the electromedical device 140 or at the medical system 100, especially at the processing module 120.

The medical system 100 according to the present invention is preferably combined with a medical alarm system. An alarm perceptible by the medical staff can be triggered hereby if the treatment recommendation 105 indicates an immediate change in or an immediate termination of the current treatment of the patient. In another variant of this exemplary embodiment, the treatment recommendation 105 is received in the presence of an alarm by reading the treatment signal 128 by the electromedical device 140 and the electromedical device 140 is controlled temporarily corresponding to the treatment recommendation 105 in order to bring the acute critical state of the patient, which is possibly indicated by the alarm, into a stable state of the patient. Such a combination of medical alarm system and a medical system according to the present invention may take place via a combination of the outputted information, i.e., the treatment recommendation 105 and the alarm, within the medical device 140 and/or within a separate alarm generation management device. The medical system 100 is not combined with such an alarm system in the exemplary embodiment shown, but it may be combined with such an alarm system. In one exemplary embodiment, not shown, such a combination is brought about by an alarm generation interface within the processing module in order to allow a communication between the processing module and the alarm generation system.

In the exemplary embodiment shown, the archiving module 110 and the processing module 120 are arranged in a common housing 150. In an alternative embodiment, the archiving module and the processing module are arranged at spaced locations from one another, especially at spaced locations and in different housings. In particular, the archiving module may be arranged centrally in a hospital and communicate with a plurality of processing modules via the corresponding archiving module interface. A central storage location can be made possible hereby for the structured patient archive.

The archiving module 110 and the processing module 120 may especially advantageously form a common device, which has access to a common processor. In this exemplary embodiment, not shown, the processing module interface would be the interface via which contents of the structured patient archive are provided for the further processing.

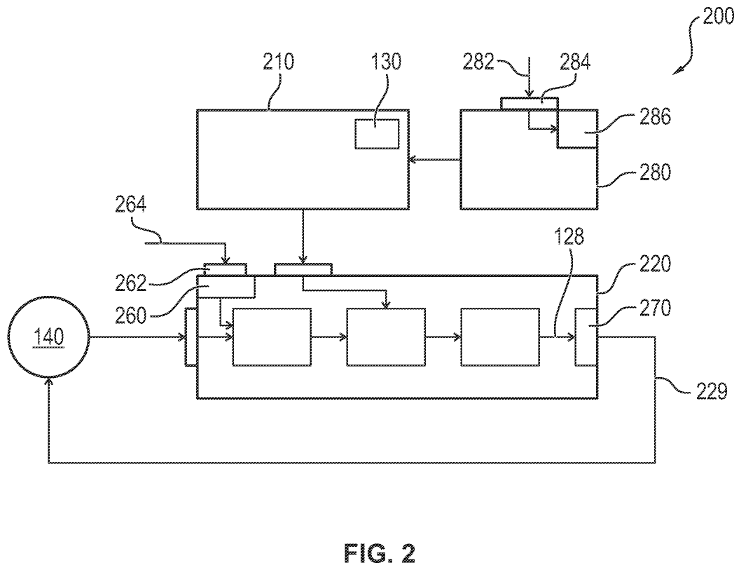

FIG. 2 shows a schematic view of a second exemplary embodiment of the medical system 200 according to the present invention.

The medical system 200 differs from the medical system 100 shown in FIG. 1 in that it has an input module 260. The input module 260 is a part of the medical system 200 according to the present invention in this exemplary embodiment shown. In one exemplary embodiment, not shown, the input module is provided outside the medical system.

The input module 260 comprises in this case an input interface 262, via which an input signal 264 is received. The input interface 262 may be, for example, a keyboard, a touchpad and/or a touch display, for the case in which the input signal 264 is formed directly by an interaction with the user of the medical system 200. In an alternative or additional exemplary embodiment, the input interface is an interface for a wireless connection or for a cable-based connection, wherein the input signal is provided by another device.

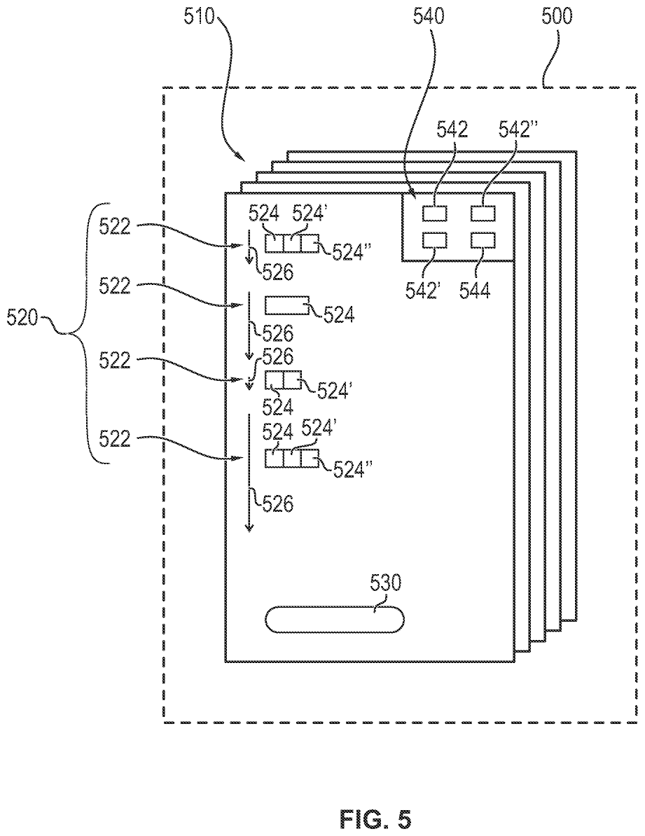

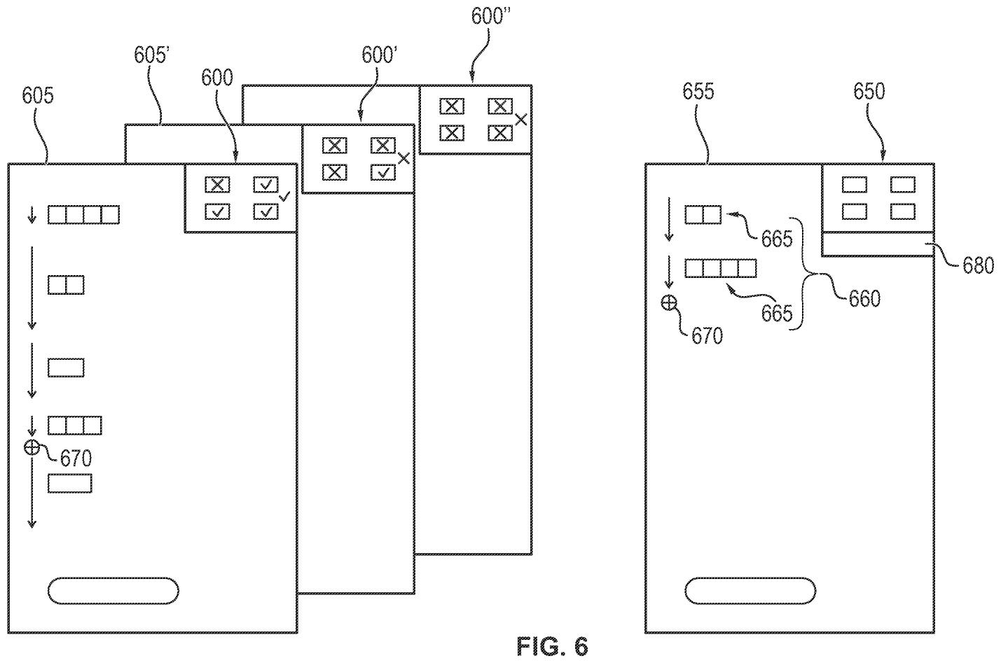

The input signal 264 comprises in this case user information, which comprises patient data to be stored in the current patient data set, a desired treatment result and/or a patient group to be assigned to the current patient data set. This user information is stored in this case together with the current patient data set. Details of the current patient data set and of the plurality of stored patient data sets are explained within the framework of FIGS. 5 and 6.

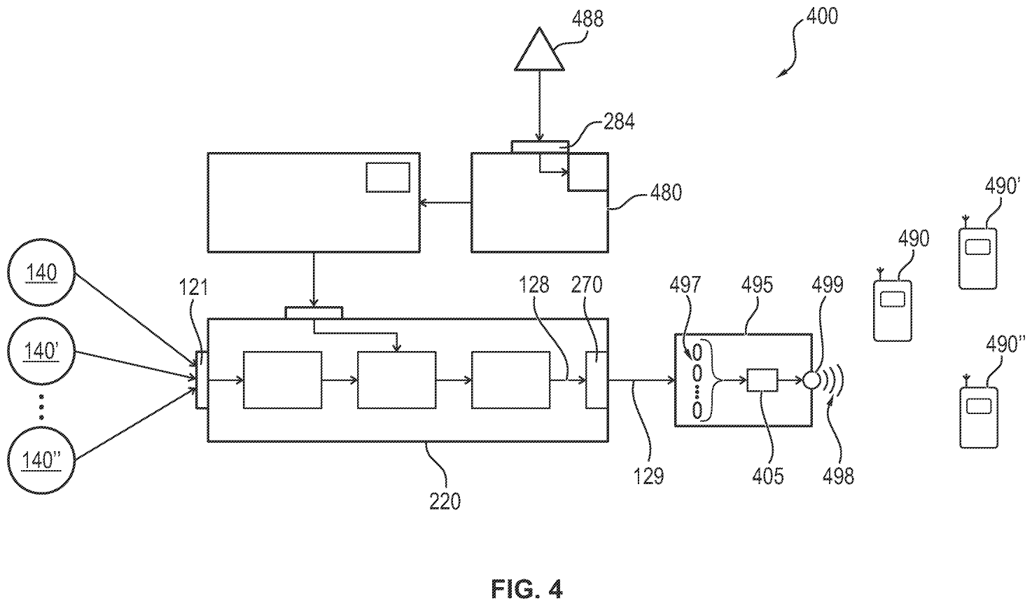

Further, the medical system 200 differs from the medical system 100 shown in FIG. 1 in that the treatment signal 128 from an output module 270 at the output of the processing module 220 is converted into a treatment signal 229 that can be received by the electromedical device 140 and is translated such that it is understandable for this device 140. After this translation into the translated treatment signal 229, the translated treatment signal 229 is outputted directly to the electromedical device 140. The communication with the electromedical device takes place here in a wireless or cable-based manner as described within the framework of FIG. 1. In the sense of the present invention and especially of the wording of the claims, the translated treatment signal 229 is also the treatment signal, because both signals transport the same content with the same treatment recommendation. The checked signal, which may indicate a different content than the treatment signal, as is explained within the framework of FIG. 4, is to be distinguished from this.

Finally, the medical system 200 also differs from the medical system 100 shown in FIG. 1 in that the data for the structured patient archive 130 are stored on a separate data collection module 280. The separate data collection module 280 is a part of the medical system 200 according to the present invention in the exemplary embodiment shown. In one exemplary embodiment, not shown, the separate data collection module 280 is arranged outside the medical system and it provides only data for the structured patient archive to the corresponding archiving module. In the exemplary embodiment, which is not shown, the data collection module forms at least partially an external patient management system, for example, of a patient care facility, e.g., of a hospital.

Patient data of earlier patients are received via a data collection signal 282 with the use of a data collection module interface 284 of the data collection module 280. Here, the data collection module interface 284 may be, for example, a keyboard, a touchpad and/or a touch display for the case in which the data collection signal 282 is generated directly by an interaction with the user of the medical system 200. In an alternative or additional exemplary embodiment, the input interface is an interface for a wireless connection or for a cable-based connection, wherein the input signal is provided by another device. The other device is preferably an external network, an external cloud or an external storage medium in this case.

The patient data of earlier patients are introduced into the memory 286, as a result of which the data bank formed thereby is updated at certain time intervals. The archiving module 210 is configured to access the data stored in the data collection module 280 in order to make them available as a structured patient archive 130 to the processing module 220. The archiving module 210 now processes the data collected on the data collection module 280 such that they can be read as a patient data set within the structured patient archive 130 for the processing module 220. The communication between the archiving module 210 and the data collection module 280 takes place in the exemplary embodiment being shown in a wireless manner, for example, via a WLAN connection, a Bluetooth connection or via a BLE connection. This communication takes place in a cable-based manner in an alternative exemplary embodiment.

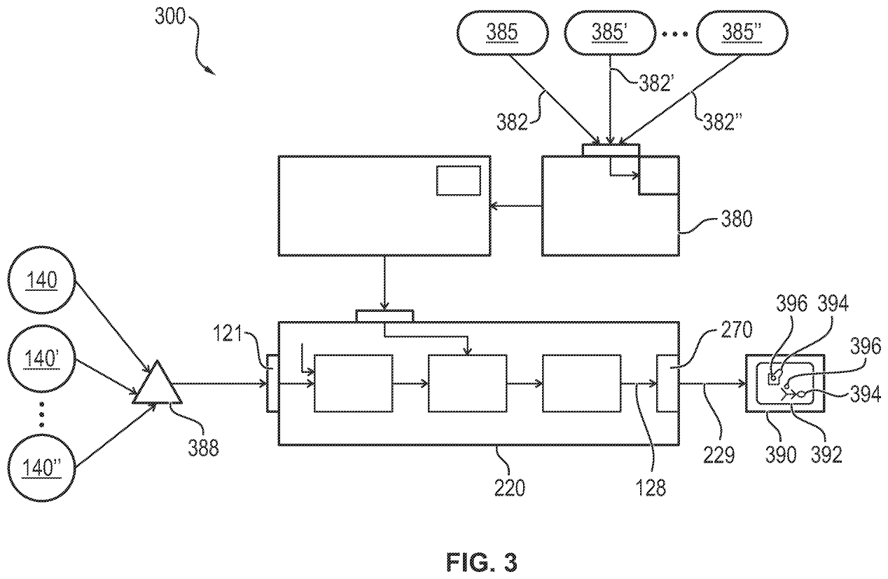

FIG. 3 shows a schematic view of a third exemplary embodiment of the medical system 300 according to the present invention.

The medical system 300 differs from the medical system 200 shown in FIG. 2 in that a plurality of additional electromedical devices 140′, 140″ are involved in a treatment of the patient in addition to an individual electromedical device 140 and they correspondingly output current patient data to the processing module 220 via the medical device interface 121. The plurality of electromedical devices 140, 140′, 140″ in this exemplary embodiment outputs the patient data to a network 388 and the network 388 communicates with the medical device interface 121. The communication takes place here preferably in a wireless manner via a WLAN connection. This communication takes place in a cable-based manner in an alternative exemplary embodiment.

Furthermore, the medical system 300 shown differs from the medical systems from the preceding exemplary embodiments in that in addition to the data collection signal 382, additional data collection signals 382′, 382″ are provided by a plurality of external data output devices 385, 385′, 385″ in order to allow the corresponding data to be stored by the data collection module 380. The data are patient data, which were recorded by different devices and were also stored within this framework on different data output devices 385, 385′, 385″.

A data output device is, for example, a medical system according to the present invention, which outputs the course of treatment and the corresponding treatment results in the form of a patient data set, preferably in an anonymized form, after a concluded treatment, for example, to a central data collection module. A respective data collection module is preferably assigned to a plurality of archiving modules according to the present invention and/or to a plurality of processing modules according to the present invention. In a preferred exemplary embodiment, the data collection module is assigned to an individual archiving module, which converts the stored data into a structured patient archive and provides this patient archive to a plurality of processing modules assigned to the individual archiving module.

A data output device may also be a storage medium, which is assigned to a patient and which stores benchmark data of a course of treatment of the corresponding patient and outputs them in an automated manner to the corresponding data collection module.

Preferably only data that were recorded within the corresponding patient care facility or which were recorded within a region of patient care facilities are used for the medical system according to the present invention.

A continuous reception of patient data of already treated patients, which patient data are processed correspondingly continuously into stored patient data sets of treated patients, is made possible by the plurality of data output devices 385, 385′, 385″. A continuously updated plurality of patient data sets can thus be stored within the structured patient archives.

Finally, the medical system 300 differs from the medical system 200 shown in FIG. 2 in that the output module 270 outputs the treatment signal 128 or the translated treatment signal 229 to an external output device 390. The external output device 390 comprises here a display 392, on which a change to be made in the current treatment is identified within the framework of a pictographic representation 394 by a corresponding marking 396.

FIG. 4 shows a schematic view of a fourth exemplary embodiment of the medical system 400 according to the present invention.

The medical system 400 differs from the medical system 300 shown in FIG. 3 in that the plurality of electromedical devices 140, 140′, 140″ communicate directly with the processing module 220 via the medical device interface 121. In addition, the data collection module interface 284 of the data collection module 480 is configured to receive patient data of already treated patients via a network 488. These patient data can be introduced into the network 488, for example, by data output devices, as they are described within the framework of FIG. 3.

Finally, the medical system 400 also differs from the exemplary embodiments described hitherto in that the output module 270 sends the treatment signal 128 directly to a testing module 495 of the medical system 400. The testing module 495 is configured to receive the treatment signal 128 or, as in this case, the translated treatment signal 229 and to compare the indicated treatment recommendation with stored device data 497 of the electromedical devices 140, 140′, 140″ and to determine a checked treatment recommendation 405 based on this comparison and to output it as a checked treatment signal 498. The output is carried out here via a wireless interface 499 of the testing module 495. The checked treatment signal 498 is preferably outputted here via a short-range wireless connection, e.g., via a Bluetooth connection or via a BLE connection. A plurality of mobile devices 490, 490′, 490″ are configured here to receive the checked treatment signal 498 via a corresponding communication interface if they are correspondingly located in the proximity of the testing module 495. In an especially preferred variant of this exemplary embodiment, only the checked treatment signal 498 from a concrete mobile device 490 for which a corresponding signature, which ensures that only authorized medical staff has access to the corresponding checked treatment recommendation, is stored in the mobile device 490.

The modules described within the framework of the above exemplary embodiments may be arranged partially in a common housing. Individual modules may also be arranged in a separate housing. Depending on the location in space of a respective module, a defined cable-based or wireless type of connection is especially advantageous. However, all prior-art types of connection shall be considered here as having been disclosed for the communication between two modules within the medical system or also for the communication of the medical system with external devices.

FIG. 5 shows a schematic view of a structured patient archive 500 for the medical system according to an exemplary embodiment according to the present invention.

The structured patient archive 500 comprises a plurality of patient data sets 510, which are based each on patient data, which were recorded in connection with an individual patient for an individual course of treatment. For reasons of data protection, it is preferably not possible to identify the particular patient involved from the patient data sets 510. Five patient data sets 510 are represented as an example in the schematic representation shown in the form of data sheets for reasons of a simple illustration. This representation is only illustrative and it does not reflect the digitized data structure within a corresponding memory.

It is shown in the exemplary representation shown that a respective patient data set 510 comprises a course of treatment 520 by the representation of individual treatment steps 522 with corresponding device settings or with an operating state 524, 524′, 524″ of an electromedical device assigned to the patient and with a respective assigned time period 526. All relevant data that are recorded in the course of a treatment of a patient are preferably acquired, especially within a concrete treatment step 522. In addition to the course of treatment 520, a treatment result 530 is assigned to the patient data set 510. The treatment result 530 may be a favorable treatment result, for example, a complete recovery, an improvement of a health status of a patient, a reduction of an exacerbation of the health status of the patient and/or a reduction of pain. The treatment result 530 may be, furthermore, an unfavorable treatment result of the patient, for example, death, an exacerbation of the health status of the patient and/or a slowdown of the recovery of the patient. It can be inferred based on the treatment result 530 whether the course of treatment 520 shown is a course of treatment that is to be emulated or whether such a course of treatment should be avoided in the future. The treatment result 530 may indicate, furthermore, a physiological final state at the end of the treatment of the patient.

Furthermore, the patient data set 510 comprises the assigned digital patient profile 540. In the exemplary embodiment shown, the patient profile 540 comprises, for reasons of clarity, only four core features, which comprise features of the course of treatment, 542, 542′, 542″, and features of the treatment result 544. In the exemplary embodiment shown, the features of the course of treatment 542, 542′, 542″ are the types of devices that were used for the treatment of the corresponding patient. In one exemplary embodiment, not shown, the patient profile comprises, furthermore, features of the stored time periods of a treatment step and/or device settings during a treatment step.

FIG. 6 shows a schematic view of a comparison of a current digital patient profile 650 with a plurality of stored digital patient profiles 600, 600′, 600″ for the medical system according to an exemplary embodiment according to the present invention.

The stored digital patient profiles 600 do not differ structurally in the exemplary embodiment shown from patient profiles 540 from the exemplary embodiments shown in FIG. 5. The selected representation is only illustrative and it does not reflect the digitized data structure within a corresponding memory.

It can, however, be seen from the representation how the features are compared within the stored digital patient profile 600 with the features of the current digital patient profile 650 of a patient who is currently to be treated. The current digital patient profile 650 is a part of a current patient data set 655 here for the patient to be currently treated. This is also the reason why only a small number of treatment steps 665 of a current course of treatment 660 are indicated in the current patient data set 655.

Furthermore, it is shown that the current treatment situation 670 of the patient to be treated is compared to the treatment situation of an already treated patient within a stored patient data set 605, if a comparison between the stored digital patient profile 600 and the current digital patient profile 650 has a sufficient agreement. A sufficient agreement is reached in the exemplary embodiment shown if three of the four features of the stored digital patient profile 600 agree with the current digital patient profile 650, consequently when, for example, the same device types are used during the treatment in the current example. In one exemplary embodiment, not shown, more features of the treatment and/or of the attending patient are stored within a respective patient profile. The type of the stored features is predefined here. An agreement in only one feature is not sufficient in this case for a sufficient agreement, as this is likewise shown within the framework of another patient data set 605′.

Furthermore, the current patient data set 655 comprises optional user information 680, for which no data were provided in this case. Separate information on the current patient may have been stored here by the input module corresponding to FIG. 2. As a result, a filtering of the stored patient data sets 605, 605′ can be carried out more rapidly, because, for example, only patient data sets of a special group of patients or of a special type of disease are compared with the current patient data set 655.

The determination of the treatment recommendation from the shown comparison of the patient profiles is preferably carried out such that the patient data sets in which the patient profiles show a sufficient agreement with the current digital patient profile are processed further. An analysis of the respective treatment results, which are stored for the selected stored patient data sets, is carried out here. For example, the courses of treatment that have led to a favorable treatment result can lead to a treatment recommendation. The courses of treatment that have led to an unfavorable treatment result may lead within the framework of the treatment recommendation to a warning that a defined mode of treatment is not promising.

For example, the effects of different medications may be stored within the stored patient data sets for a similar treatment situation. Thus, the effect of a concrete medication can be analyzed over a plurality of random samples and the medication that is most promising for the current health status of the patient and for the current treatment situation can be identified hereby. As an alternative or in addition, it is possible, for example, to analyze the effect of different ventilation modes of a ventilator over a plurality of random samples and to identify thereby the most promising ventilation mode for the current health status of the patient and for the current treatment situation.

In an especially preferred exemplary embodiment, the determination of the treatment recommendation is carried out by the processing module based on the processing of the patient data sets suitable in view to the comparison of the patient profiles by a neuronal network or by a prior-art machine learning process. A reliable treatment recommendation can thus be determined especially efficiently for the current patient by means of a large data base. The corresponding algorithm, which is capable of learning, now learns from the suitable patient data sets identified by said comparison.

The processing module preferably indicates if no suitable data base or an excessively small data base of suitable patient data sets is available for the current treatment situation.

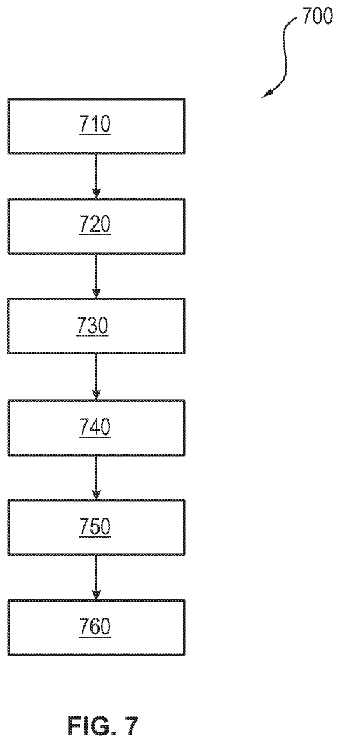

FIG. 7 shows a flow chart of an exemplary embodiment of a process 700 according to another aspect of the present invention.

The process 700 according to the present invention is configured to provide a treatment recommendation. It has the process steps explained below.

A first step 710 comprises the provision of a structured patient archive, in which a respective patient data set is stored for each patient of a plurality of patients, wherein the respective patient data set indicates a course of treatment and a treatment result of the corresponding patient and wherein at least one digital patient profile, which indicates features of the course of treatment and of the treatment result, is assigned to the patient data set of a respective patient.

A next step 720 comprises a reception of current patient data recorded by the medical device.

A next step 730 comprises the determination of a current patient data set with a corresponding current digital patient profile from the current patient data recorded.

A further step 740 comprises a comparison of the current digital patient profile with the plurality of stored digital patient profiles.

A next step 750 comprises the determination of a treatment recommendation based on the preceding comparison.

A final step 760 comprises the outputting of a corresponding treatment signal, wherein the treatment signal indicates the determined treatment recommendation.

The first step 710 is carried out such that it is repeated at certain time intervals, because the structured patient archive is regularly updated by new patient data sets. As a result, an updated structured patient archive is repeatedly provided within the framework of step 710.

Steps 720 and 730 are preferably carried out one after another, and the current patient data set is always updated by the received patient data. In an alternative exemplary embodiment, the patient data set is determined from the received patient data at predefined time intervals or triggered by a corresponding triggering signal, for example, by a user input.

Based on the patient data set determined in step 730, steps 740 and 750 are carried out one after another. Thus, the comparison between the current digital patient profile and stored digital patient profiles, as described in the preceding exemplary embodiments, leads directly to the determination of the treatment recommendation. Step 750 can be carried out here with the use of a prior-art neuronal network or by prior-art machine learning processes based on the comparison from step 740 in the already described manner.

The final step 760 is preferably triggered directly by the determination of the treatment recommendation. As an alternative, the triggering of step 760 may depend on the content of the treatment recommendation. For example, a treatment recommendation, which indicates a continuation of a current treatment, can thus lead to a suppression of the output of the treatment signal in order not to output redundant and/or unnecessary information. Furthermore, an output of the treatment signal may take place in this alternative exemplary embodiment only when a minimum deviation between the current treatment and a treatment recommended within the framework of the treatment recommendation is exceeded. The minimum deviation corresponds here to a deviation of a concrete setting value, for example, of the electromedical device, e.g., a drug dosage, of an event rate of a treatment event and/or of an alarm limit.

The process 700 according to the present invention is preferably carried out by a single processor, which can access corresponding memory areas during the processing of data. As an alternative, the process may be carried out on different devices, and individual process steps may be carried out separated in space from other process steps. Thus, the provision according to step 710 may be carried out, for example, separated in space from the other steps of the process 700.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300, 400 Medical system
105 Treatment recommendation
110, 210 Archiving module
112 Memory of the archiving module
120, 220 Processing module
121 Medical device interface
122 Archiving module interface
124, 655 Current patient data set
125, 650 Current digital patient profile
126 Comparison
128 Treatment signal
130, 500 Structured patient archive
132, 510, 605 Stored patient data set
134, 540, 600, 600′, 600″ Stored patient profile
140, 140′, 140″ External medical device
142 Patient data
150 Housing
229 Translated treatment signal
260 Input module
262 Input interface
264 Input signal
270 Output module
280, 380, 480 Data collection module
282, 382, 382′, 382″ Data collection signal
284 Data collection module interface
286 Memory of the data collection module
385, 385′, 385″ Data output device
388, 488 Network
390 Output device
392 Display
394 Pictographic representation
396 Marking
405 Checked treatment recommendation
490, 490′, 490″ Mobile device
495 Testing module
497 Device data
498 Checked treatment signal
499 Wireless interface
520, 660 Course of treatment
522, 665 Treatment step
524, 524′, 524″ Operating state
530 Treatment result
542, 542′, 542″ Feature of the course of treatment
544 Feature of the treatment result
670 Treatment situation
680 Optional user information
700 Process
710, 720,730, 740, 750, Process steps
760

What is claimed is:

1. A treatment recommendation medical system comprising:
an archiving module configured to provide a structured patient archive comprising a respective patient data set stored for each patient of a plurality of patients, wherein the respective patient data set indicates a course of treatment and a treatment result of a corresponding patient, and at least one digital patient profile, which indicates features of the course of treatment and of the treatment result, is assigned to the patient data set of a respective patient;
a processing module comprising a medical device interface and an archiving module interface, wherein the processing module is configured:
to receive current patient data recorded by an external medical device via the medical device interface and to determine therefrom a current patient data set with a corresponding current digital patient profile;
to compare the current digital patient profile with the plurality of stored digital patient profiles by communication with the archiving module via the archiving module interface;
to determine a treatment recommendation based on the comparison, taking into consideration the treatment results indicated by the stored digital patient profiles; and
to output a treatment signal, wherein the treatment signal indicates the determined treatment recommendation.

2. A treatment recommendation medical system in accordance with claim 1, wherein the determination of the treatment recommendation comprises an analysis of the stored patient data sets in which the digital patient profile has a sufficient agreement with the current digital patient profile.

3. A treatment recommendation medical system in accordance with claim 2, wherein the analysis of the stored patient data sets in which the digital patient profile has a sufficient agreement with the current digital patient profile comprises an analysis of a relationship between the respective course of treatment indicated by the corresponding patient data set and the respective treatment result indicated by the corresponding course of treatment.

4. A treatment recommendation medical system in accordance with claim 1, wherein each stored patient data set comprises at least one operating state of an electromedical device assigned to the corresponding patient.

5. A treatment recommendation medical system in accordance with claim 1, wherein:
respective time periods of a treatment step are assigned to the course of treatment within a respective stored patient data set; and
an analysis of the assigned time periods is carried out to determine a treatment recommendation.

6. A treatment recommendation medical system in accordance with claim 1, wherein the treatment recommendation comprises a treatment warning.

7. A treatment recommendation medical system in accordance with claim 1, wherein the treatment recommendation comprises at least one of:
an operating state of an electromedical device assigned to the corresponding patient, which operating state is to be used;
a relative change in the operating state of an electromedical device assigned to the corresponding patient;
an electromedical device to be newly assigned to the corresponding patient;
a duration of a treatment to be carried out; and
a frequency of a treatment to be carried out.

8. A treatment recommendation medical system in accordance with claim 1, wherein the archiving module is connected to an external patient management system such that patient data sets of treated patients are stored continuously in the structured patient archive.

9. A treatment recommendation medical system in accordance with claim 1, wherein the medical system further comprises an input module configured to receive user information via an input interface, wherein the user information comprises at least one of:
patient data to be stored in the current patient data set;
a desired treatment result; and
a patient group to be assigned to the current patient data set.

10. A treatment recommendation medical system in accordance with claim 1, further comprising a testing module configured:
to receive the treatment signal and to compare the indicated treatment recommendation with device data of at least one electromedical device assigned to the corresponding patient;
to determine a checked treatment recommendation based on this comparison; and
to output a checked treatment signal, wherein the checked treatment signal indicates the checked treatment recommendation.

11. A treatment recommendation medical system in accordance with claim 1, further comprising an output module configured to output at least one of the treatment signal and the checked treatment signal to at least one electromedical device assigned to the corresponding patient.

12. A process for providing a treatment recommendation, the process comprising the steps of:
providing a structured patient archive comprising a respective patient data set stored for each patient of a plurality of patients, wherein the respective patient data set indicates a course of treatment and a treatment result of the corresponding patient, and wherein at least one digital patient profile, which indicates features of the course of treatment and of the treatment result, is assigned to the patient data set of a respective patient;
receiving current patient data recorded by a medical device;
determining a current patient data set with a corresponding current digital patient profile from the recorded current patient data;
comparing the current digital patient profile with the plurality of stored digital patient profiles;
determining the treatment recommendation based on the comparison, taking into consideration the treatment results indicated by the stored digital patient profiles; and
outputting a corresponding treatment signal, wherein the treatment signal indicates the determined treatment recommendation.

13. A process in accordance with claim 12, further comprising the steps of:
receiving the treatment signal and comparing the indicated treatment recommendation with device data of at least one electromedical device assigned to the corresponding patient; and
determining a checked treatment recommendation based on the preceding comparison and outputting of a corresponding checked treatment signal, wherein the checked treatment signal indicates the checked treatment recommendation.
